(19) United States
(12) Patent Application Publication
(10) Pub. No.: US 2021/0327612 A1
(43) Pub. Date: Oct. 21, 2021

(54) SYSTEM, METHOD, KIT AND APPARATUS FOR ORGANIZING CABLES

(71) Applicants: Susan K. Park, Dublin, OH (US); Robyn E. Sattelmyer, Dublin, OH (US)

(72) Inventors: Susan K. Park, Dublin, OH (US); Robyn E. Sattelmyer, Dublin, OH (US)

(21) Appl. No.: 17/234,440

(22) Filed: Apr. 19, 2021

Related U.S. Application Data
(60) Provisional application No. 63/012,473, filed on Apr. 20, 2020.

(51) Int. Cl. H01B 7/36; H01B 7/00; A61L 2/18

(52) U.S. Cl. CPC H01B 7/368; H01B 7/0045; A61L 2202/24; A61L 2202/15; A61L 2/18

(57) ABSTRACT

An apparatus, system and method for organizing and identifying cables can include cable organizers such as hollow, tubular cylinders. Each cable organizer can slidably and releasably engage a subset of the cables simultaneously. Each cable organizer can be freely movable along a length of the subset of the cables under the influence of an externally applied force; released and held in place via static friction. In addition, unique identifiers can be attached and/or labeled on respective ones of the cable organizers to separately identify each cable and/or subset of bundled cables.

### CROSS-REFERENCE TO RELATED APPLICATIONS

This application claims the benefit of U.S. Provisional Application Ser. No. 63/012,473, filed Apr. 20, 2020, which is incorporated herein by reference in its entirety.

### TECHNICAL FIELD

This application generally relates to organizers and, in particular, to a system, method, kit and apparatus for cables organization, and in particular, to cables used in a medical arena.

### BACKGROUND

Some devices, such as an electrocardiogram (EKG) machine, can have numerous leads, also referred to as wires or cables, that often become tangled and confused with one another, particularly if the cables are of random or the same color and otherwise unmarked. These problems can cause longer treatment times, incorrectly positioning the EKG attachments on the patient, erroneous testing data, and potentially unnecessary or withheld medical treatments. Such events are costly and create potentially harmful delays, re-testing unnecessary investigations, admission to hospital and improper treatment.

Most EKG machines and cables connected thereto do not have any form of a cable management system. There are a number of aftermarket products available, however, none have gained widespread adoption due to their lack of functionality and in some cases, excessive cost due to their complexity of design. For example, drawbacks of conventional cable organizers include: the cables, upon being routed through the cable organizer, are too short to reach the appropriate areas of placement; they do not provide an ability for grouping (bundling) cables together; they must be removed in order to clean the cables; and if the cables are moved or pulled during placement the cables inadvertently pop out of the organizer.

Although conventional solutions provide some benefits, improvements in cable organizers continue to be of interest to resolve at least the drawbacks discussed above.

### SUMMARY

This section provides a general summary of the disclosure and is not intended to be considered a complete and comprehensive listing of the disclosure's full scope or all of its aspects, advantages, objectives and/or features.

It is an object of the present disclosure to provide a system, method, kit and apparatus for organizing cables that addresses at least those issues discussed above of known cable organizers.

Embodiments of a system, method, kit and apparatus for organizing and identifying cables (also referred to as wires or leads, wherein the terms cable, wire, and lead are intended to have the same meaning herein, though, as known, a cable can be comprised of multiple, bundled wires or leads), particularly cables in a medical arena, is disclosed. For example, versions of a cable organizer constructed in accordance with the disclosure herein can organize and identify the cables of equipment within a medical arena, such as those directed to IV drip bags and an electrocardiogram machine (known as an ECG or EKG machine), by way of example and without limitation. The cable organizer can include one or more hollow tubes that can individually enclose and capture two or more EKG cables in grouped, also referred to as bundled, relation with one another, or be disposed on select ones of cables to identify the individual cable. The cable organizer can be disposed about predetermined, selected cables such that the selected cables can be grouped and held and routed together based upon user preference. The cable organizer(s), being tubular, can be intentionally slid along the pre-selected grouped cables, while in use, to avoid creating an obstruction, such as while performing a test on a patient, yet be easily slid back to any desired location along the length of the grouped cables, such as may be desired while transporting the patient from one location to another, to prevent tangling of and damage to the cables and/or equipment connected thereto.

In accordance with another aspect, although the cable organizers can be readily moved along a length of the cable being organized and/or identified, upon removing an externally applied force needed to slide the cable organizers, sufficient static friction between the cable organizers and the cables being grouped functions to inhibit inadvertent movement of the cable organizers relative to the grouped cables, regardless of the special orientation of the grouped cables, thereby maintaining the cable organizers in their desired relative location along the grouped cables.

In accordance with a further aspect, the cable organizer can include a mechanism to sanitize or otherwise apply a treatment to an outer surface of the cable(s) extending therethrough as the cable(s) are moved through in internal cavity of the tubular cable organizer. The cable organizer can improve efficiency by keeping the cables grouped by strategic placement on or relative to the patient, as well as decrease the potential for committing errors through organization of the numerous cables (e.g., color coding and/or notation on the cable organizer(s)) to reduce the potential of misplacement of the proper cable to the associated piece of medical testing equipment. Accordingly, the individual cables, with the assistance of indicator(s) on the cable organizers, are assured of being operably connected to the correct equipment/electrical receptacles.

In accordance with a further aspect, a cable organizer constructed in accordance with the disclosure herein is economical in manufacture and use, thereby minimizing the cost associated with the use thereof, while ultimately reducing the total potential cost of a medical procedure by reducing the potential of error and damage to cables and/or equipment attached to the cables and by reducing the amount of time needed to ensure proper connections are made between the cables and the desired pieces of medical equipment and electrical outlets.

In accordance with a further aspect, a cable organizer constructed in accordance with the disclosure herein can be readily discarded after use, given the relatively low cost of manufacture thereof, thereby negating the need for cleaning and sterilization thereof between uses. Accordingly, the cable organizers are well suited for one time, disposable use.

In accordance with another aspect, a method of organizing and identifying cables is provided herein. The method includes disposing a cable organizer about a plurality of cables to bundle the plurality of cables together. The method further includes sliding the cable organizer along a length of the bundled cables via application of an externally applied force sufficient to overcome static friction between the cable organizer and an outer surface of the bundled cables, and then releasing the cable organizer when in the desire location along the bundled cables, whereupon the static friction between the cable organizer and an outer surface of the bundled cables maintains the cable organizer in the desired location regardless of the spacial orientation of the bundled cables, even if extending in a vertical orientation.

In accordance with another aspect, the method can include providing an outer surface of the cable organizer with a textured surface facilitating grip while locating the organizer along the length of the bundled cables being organized, as well as facilitating marking the outer surface with an identifying marking, such as with a writing implement.

In accordance with another aspect, the method can include providing the cable organizer having a predetermined color to facilitate organizing and identifying cables relative to one another.

In accordance with another aspect, a system for organizing and identifying cables extending from a source to a target is provided. The system includes a plurality of cable organizers, with each cable organizer configured to slidably and releasably engage a cable and/or a subset of the cables simultaneously, such that each cable organizer is freely movable along a length of the subset of the cables under application of an externally applied force, while the cable organizer remains in a releasably fixed location via static friction against the cable(s) upon removal of the externally applied force. Further, a cleaning agent on an inner surface of each cable organizer can be configured to sanitize the cable(s) while the cable organizer is being moved relative to the cable(s).

The foregoing and other objects, features and advantages of the non-limiting example embodiments disclosed herein will be apparent to those of ordinary skill in the art in view of the following detailed description, taken in conjunction with the appended claims and the accompanying drawings.

### BRIEF DESCRIPTION OF THE DRAWINGS

So that the manner in which the objects, features and advantages of the embodiments are attained and can be understood in more detail, a more particular description can be had by reference to the example embodiments thereof that are illustrated in the appended drawings. However, the drawings illustrate only example embodiments and therefore are not to be considered limiting in scope as there can be other equally effective embodiments falling within the scope of the claims.

FIG. 1 illustrates a medical arena with a plurality of cable organizers disposed about cables associated with medical equipment to organize and/or identify the cables to inhibit tangling and improper routing/connection of the cables to the proper electrical receptacle;

FIG. 2A illustrates a cable organizer of FIG. 1 constructed in accordance with the disclosure disposed about a plurality of cables to bundle and organized the cables with one another;

FIG. 2B illustrates a cable organizer of FIG. 1 constructed in accordance with the disclosure disposed about single cables to facilitate identifying and distinguishing the cables from one another;

FIG. 3 is a perspective view of the cable organizer of FIGS. 2A and 2B;

FIG. 4 is an end view of the cable organizer of FIG. 3 looking generally along the direction of arrow 4;

FIG. 5 is a cross-sectional view of the cable organizer of FIG. 3 taken generally along the line 5-5;

FIG. 6 is a perspective view of a cable organizer constructed in accordance with another aspect of the disclosure;

FIG. 7 is a cross-sectional view of the cable organizer of FIG. 6 taken generally along the line 7-7;

FIG. 8 is a perspective view of a cable organizer constructed in accordance with another aspect of the disclosure;

FIG. 9 is a cross-sectional view of the cable organizer of FIG. 8 taken generally along the line 9-9;

FIG. 10 is a perspective view of a cable organizer constructed in accordance with another aspect of the disclosure;

FIG. 11 is a cross-sectional view of the cable organizer of FIG. 10 taken generally along the line 11-11;

FIG. 12 is a perspective view of a cable organizer constructed in accordance with another aspect of the disclosure;

FIG. 13 is a cross-sectional view of the cable organizer of FIG. 12 taken generally along the line 13-13;

FIG. 14 is a perspective view of a cable organizer constructed in accordance with another aspect of the disclosure;

FIG. 15 is a cross-sectional view of the cable organizer of FIG. 14 taken generally along the line 15-15;

FIG. 16 is a perspective view of a cable organizer constructed in accordance with another aspect of the disclosure;

FIG. 17 is a cross-sectional view of the cable organizer of FIG. 16 taken generally along the line 17-17;

FIG. 18 is a perspective view of a cable organizer constructed in accordance with another aspect of the disclosure; and

FIG. 19 is a cross-sectional view of the cable organizer of FIG. 18 taken generally along the line 19-19.

The use of the same reference symbols in different drawings indicates similar or identical items.

### DETAILED DESCRIPTION OF EXAMPLE EMBODIMENTS

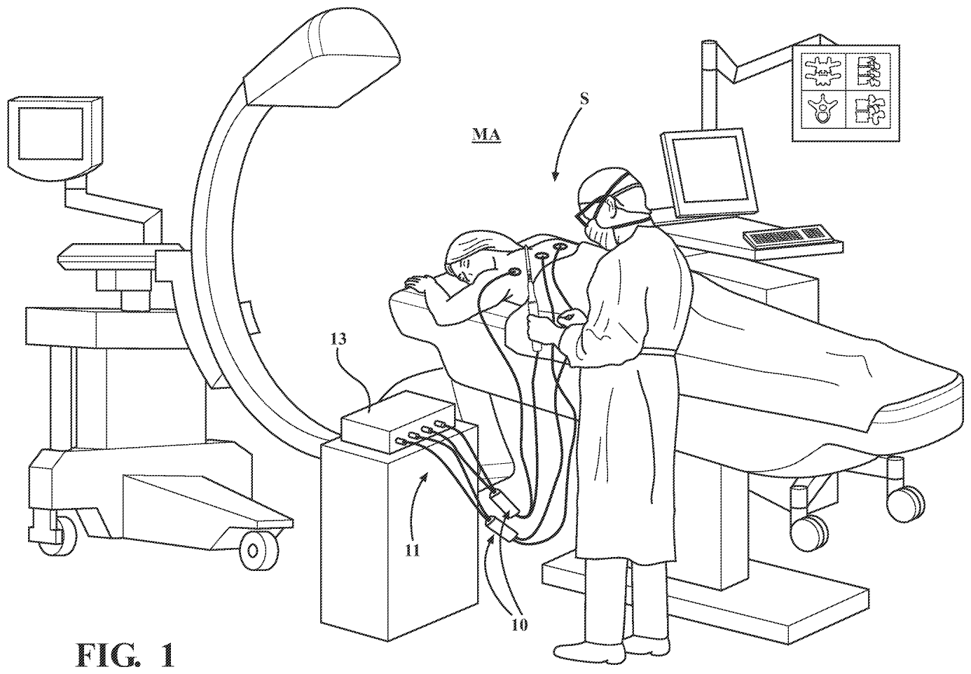

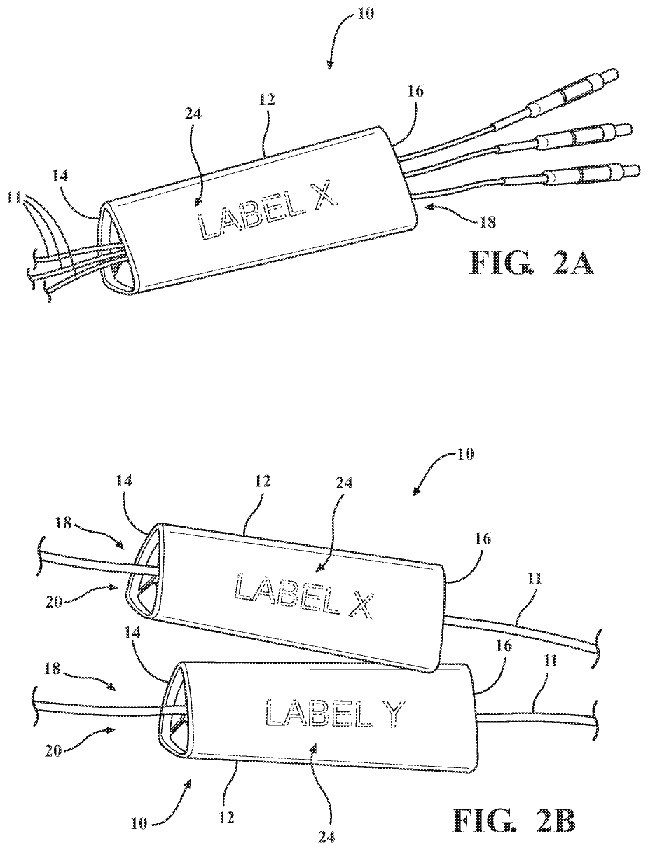

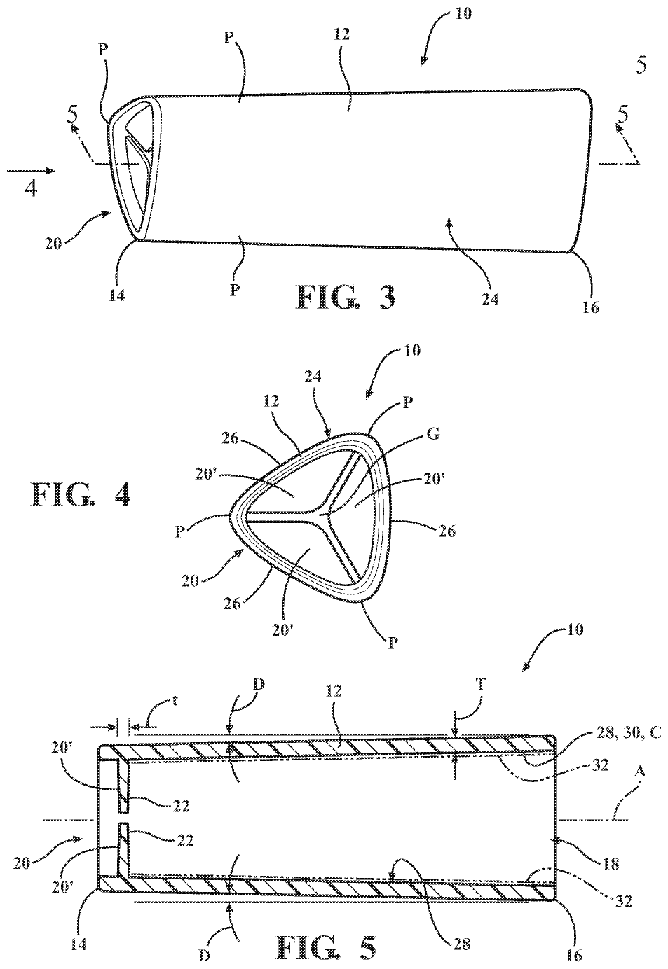

FIGS. 1-19 depict various embodiments of a system S, method, kit and apparatus for organizing cables, tubes, cords and the like, referred to hereafter as cables and/or tubes 11, with a particular focus being on cables, tubes, and cords of a medical apparatus 13 in a medical arena MA (FIG. 1). For example, an apparatus, referred to hereafter as cable organizer 10, can be provided to save time and expense by keeping EKG electrical leads (cables) 11 organized to prevent misplacement, tangles, and to expedite proper placement. Benefits can include, in some versions, cables 11 remains tangle free and can be grouped according to preference; emergent EKGs are not delayed due to tangled cables; cable placement errors are reduced or eliminated; and easy sanitation of the cables 11 can be provided via cleaning/sterilizing agent(s) integral to the cable organizer 10. Some examples include cylindrically or generally cylindrically (meaning an outer surface of the cable organizer 10 may be other than truly cylindrical, such as having a taper, also referred to as draft, or flared surface(s)) shapes to prevent tangling, enhance handling and movement along a fully length of the cables to a distal end of the cables for attachment to a patient. Various embodiments can be colored, or otherwise identified to assist in identification of the desired cables and/or tubes 11 and prevent positioning errors on the patient. Cables 11 of a similar placement location can be grouped (bundled) together for faster identification, thereby improving the rate of placement and testing while also reducing errors. Embodiments can include a non-porous hard surface that is easy to clean, durable and protects the cables 11, along with a textured surface to facilitate writing on the surface with a writing implement to facilitate proper identification of the associated cable 11. In one medicine organizing example, a cable organizer 10, approximately 0.5 to 1 inch in diameter and 3 inches in axial length, by way of example and without limitation, can include different colors to signify medication classifications, such as from IV drip bags. The medical name of the medication can be included on one side and a generic (common name) can be included on the other side, if desired. Space for including a date and time, as well as the initials of the user can be provided as well. The drug classification also can be included on the cable organizer 10. Each of the above is generally identified by Labels X and Y in FIGS. 2A, illustrating a plurality of cables 11 organized and bundled with one another in a common cable organizer 10, wherein each of the cables 11 can be routed to the same piece of equipment, thereby keeping the cables 11 of the same piece of equipment grouped for ease and accuracy of attachment thereto, if desired, and 2B, illustrating a single cable 11 extending through a cable organizer 10, wherein the cable organizer 10 can be colored, labeled, or otherwise identified to enable easy identification of the cable/tube 11 extending through the cable organizer 10.

An example embodiment can be color coded as follows, in some versions: black (steroid) dexamethasone, Decadron, methylprenisolone, Solu-Medrol; purple (insulin) Humulin, Lispro; red (cardiac) Diltiazem, Cardizem, Adenosine, Adenocard; orange (opiod) morphine, fentanyl; white (hydration) dextrose, lactated ringer's; yellow (antibiotic) Cefriaxone, Rocephin, Efdinir, Omnicef; grey (electrolyte) calcium, magnesium; blue (anti-coagulants) Heparin, Aggrenox; green (paralytic) Etomidate, Haloperidol; and pink (blood product) Albumin, PRBC.

In one embodiment, kit for organizing and identifying cables 11 can include cable organizers 10 and, unique identifiers, such as pre-identified adhesive labels, which can be attached to respective ones of the cable organizers 10 to separately identify each subset of the cables 11, and/or a variety of differently colored writing implements can accompany the cable organizers 10 for writing on an outer surface of the cable organizers 10, as desired. Otherwise, it is to be understood that labels or any other decorative, stylized features can be applied in a mold during a molding process used to form the cable organizer 10 and that identifying features can be applied via pad printing and/or any other known mechanism for marking a surface.

In another embodiment, a system for organizing and identifying cables can extend from a source (e.g., an EKG machine) to a target (e.g., a patient). The system S can include a plurality of cables organizers 10. Each cable organizer 10 can be formed from a resilient polymeric material, with each cable organizer 10 being configured to slidably and releasably engage a plurality of the cables and/or tubes 11 simultaneously to form separate bundles of the cables and/or tubes 11. Each cable organizer 10 can be freely movable along a length of the bundled cables/tubes 11 via application of a suitable externally applied force sufficient to overcome an intended, built-in static friction between the cables/tubes 11 an inner surface of the cable organizers 10. In addition, an inner surface of the cable organizer 10 can include a cleaning agent 30 configured to sanitize the cables and/or tubes 11, as discussed further below.

Cable organizers 10 constructed in accordance with the disclosure and systems S established therewith can differ from each other by at least one physical parameter. For example, the physical parameter can be at least one of color, shape, size or outer surface texture. In some embodiments, the physical parameter can be a labeling system to distinguish the cable organizers 10 from one another, thereby allowing a user to selectively locate and identify the cables and/or tubes 11. Examples of the labeling system can include labels that are configured to wrap around and be secured to an outer surface of respective ones of the cable organizers 10.

Embodiments of the cable organizer 10 can include at least one of an external grip feature, handle or texture to facilitate manual, slip-free manipulation by a user. In one embodiment, as shown in FIGS. 2A-5, each cable organizer 10 comprises a generally cylindrical wall 12 having a circumferentially solid, closed shape, thereby being tubular, and extending along a longitudinal axis A (FIG. 5) between opposite ends 14, 16. The wall 12 bounds an internal passageway, also referred to as cavity 18, which extends from one end 14 to the opposite end 16. In an example embodiment, the cable organizer 10 can have an axial length of about 1 to 6 inches, and in one preferred embodiment, between about 2 to 4 inches, with the cavity 18 having an inner radial dimension between about 0.5 to 2 inches, and in one preferred embodiment, between about 0.5 to 1 inches. Collectively, each bundle of cables 11 preferably has an outer radial dimension that is less than the inner radial dimension of the cavity 18, thereby facilitating slidable movement of the cable organizer 10 along the length of the bundled cables 11, though, the cable organizer 10 has a retention feature 20 to inhibit relative movement between the cable organizer 10 and the bundled cables 11 upon locating and releasing the cable organizer 10.

As shown in FIG. 4, the retention feature 20 of the cable organizer 10 is provided by a plurality of flaps, also referred to as fingers 20′. The fingers 20′ extend radially inwardly from the wall 12 to free ends 22. The free ends 22 (FIG. 5) of the fingers 20′ and the entire periphery of the fingers 20′ are spaced from one another by a gap G (FIG. 4), such that adjacent fingers 20′ are spaced from one another by the gap G. With the wall 12 being shown having a generally triangular outer surface 24, with convex surfaces 26 extending between three peaks P, three fingers 20′ are provided, with each finger 20′ extending radially inwardly from a respective one of the convex, arcuate surfaces 26. The three fingers 20′ are configured in coplanar relation with one another. The fingers 20′ are able to flex resiliently independently from one another to facilitate disposal of the cable organizer 10 about the cables 11, wherein the fingers flex axially in a first direction while sliding the cable organizer 10 along the cables 11 in a first direction, and flex axially in a second direction opposite the first direction while sliding the cable organizer 10 along the cables 11 in a second direction opposite the first direction. With the fingers 20′ being resilient, they remain in engagement with the cables 11 during and after assembly, thereby resulting in static friction between the free ends 22 of the fingers 20′ and the outer surfaces of the cables 11. The static friction is intended to be sufficient to inhibit relative movement between the cable organizer 10 and the bundled cables 11 absent a sufficient externally applied force, such as may be imparted to slide the cable organizer 10 as desired. Accordingly, upon locating the cable organizer 10 in the desired position along the cables 11, the cable organizer 10 remains in the desired position until intentionally moved via application of an externally applied force. To facilitate gripping and sliding the cable organizer 10, at least one or more of the convex surfaces 26 extending between the peaks P can be formed having a textured, also referred to as knurled, surface finish. The textured surface finish also facilitates marking the outer surface with a writing implement, as desired, to enhance the ability to distinguish cables 11 from one another. It is further contemplate that self-adhesive labels can be adhered to one or more of the convex surfaces 26 to facilitate distinguishing and identifying the cables 11 from one another.

To further facilitate gripping the cable organizer 10, as well as to facilitate manufacture, thereby reducing cost, the outer surface 24 can be formed having a slight taper, also referred to as draft D, extending from one end 14 to the opposite end 16. The draft D can be formed having a constant taper relative to the axis A from one end 14 to the other end 16 and forms an angle greater than 0 degrees and less than about 10 degrees, and more preferably less than about 5 degrees relative to the axis A. As such, the outer maximum radial dimension of the end 14 is different from the outer maximum radial dimension of the end 16, wherein the end 14 having the retention feature 20 is shown having an outer maximum radial dimension that is less that the outer maximum radial dimension of the end 16. Accordingly, the retention feature 20 is proximate the end 14 with the reduced outer maximum radial dimension. The draft D of the outer surface 24 facilitates grip and manufacture of the cable organizer 10.

To enhance the ability of the fingers 20′ to flex in opposite axial directions during assembly and while repositioning the cable organizer 10 along the length of the cables 11, when desired, the fingers 20′, as shown in FIG. 5, can be provided having a constant finger thickness (t) that is less than a constant wall thickness (T) of the wall 12. Accordingly, while the wall thickness (T) can be provided having a sufficient thickness to provide stability and rigidity to the bundled cables 11, such as between about 0.040-0.070 inches, and in one example embodiment between about 0.050-0.060 inches, the fingers 20′ can be provided having a finger thickness between about 0.020-0.030. As such, the fingers 20′, while acting to locate the cable organizer 10 relative to the cables 11, remain resiliently flexible for sake of assembly, relocation and removal.

In accordance with a further aspect of the disclosure, as shown in FIG. 5, an inner surface 28 of the cable organizer 10 can be provided having a cleaning and/or sterilizing agent, referred to hereafter as sanitizer 30, configured to facilitate cleaning an outer surface of the cable(s) 11 while sliding the cable organizer 10 therealong. The sanitizer 30 can be deposited on at least a portion or the entirety of the inner surface 28 via any suitable mechanism, including via formation of a coating C in a coating process. Otherwise, the sanitizer 30 can be disposed on the inner surface via a treated substrate, such as a thin absorbent film or scrim 32, such as sponge-like scrim material, by way of example and without limitation. The sanitizer 30 can be configured to be manually released by a user by sliding the cable organizer 10 along the cables 11. In accordance with another aspect, the cable organizer 10 can be configured to be squeezed by the user to release the sanitizer 30. Examples of the cleaning agent can comprise at least one disinfectant, germicidal agent, anti-microbial agent, alcohol sponge, quaternary ammonium, hypochlorite, phenolic, peracetic acid, accelerated hydrogen peroxide, bleach, chlorine releasing agent, sodium hypochlorite or iodophor. The cleaning agent can be contained within pores of the substrate 32 or adhered directly to the inner surface 28. The cleaning agent can be configured to be manually released by a user of the system. For example, the wall 12 can be configured to be squeezed by the user to release the cleaning agent.

The cable organizer 10 can be formed of any suitable polymeric material, with some presently preferred materials including polyethylene terephthalate (PET) and polypropylene (PP). Regardless, the polymeric material used, the cable organizer 10 is preferably molded, such as in a low cost injection molding operation. Accordingly, the cable organizer 10 can be made in generally low cost fashion, thereby lending to its being disposable, if desired.

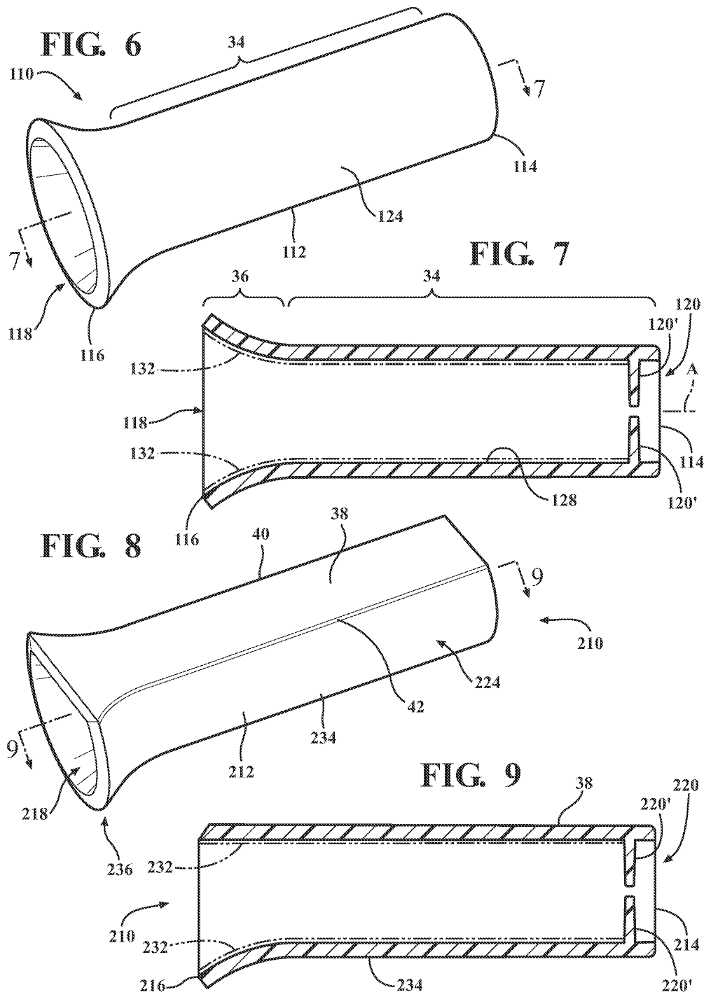

In FIGS. 6 and 7, a cable organizer 110 constructed in accordance with another aspect of the disclosure is shown, wherein the same reference numerals as used above, offset by a factor of 100, are used to identify like features.

Cable organizer 110 has an annular wall 112 with a circumferentially solid, closed shape, thereby being tubular, and extending along a longitudinal axis A (FIG. 7) between opposite ends 114, 116. The wall 112 has an outer surface 124 and an inner surface 128 that bounds a cavity 118, which extends from one end 114 to the opposite end 116. The outer surface 124 has a constant radius portion 34 extending from one of the ends 114 toward the other of the ends 116 and a flared portion 36 extending from the other of the ends 116 to the constant radius portion 34. The constant radius portion 34, in one non-limiting embodiment, is cylindrical. A retention feature 120 is provided by a plurality of fingers 120′ proximate the end 114 opposite the flared end 116. Retention feature 120 is the same as discussed above for retention feature 20, and thus, no further description is needed. Flared end 36 facilitate gripping and moving cable organizer 110 along a cable bundle. Outer surface 124 can be textured, and inner surface 128 can include a sanitizer 132, if desired, as discussed above.

In FIGS. 8 and 9, a cable organizer 210 constructed in accordance with another aspect of the disclosure is shown, wherein the same reference numerals as used above, offset by a factor of 200, are used to identify like features.

Cable organizer 210 is similar to cable organizer 110, having an annular wall 212 extending along a longitudinal axis A (FIG. 9) between opposite ends 214, 216. The wall 212 has an outer surface 224 and an inner surface 228 that bounds a cavity 218, which extends from one end 214 to the opposite end 216. The outer surface 224 has a constant radius portion 234 extending from one of the ends 214 toward the other of the ends 216 and a flared portion 236 extending from the other of the ends 216 to the constant radius portion 234. A retention feature 220 is provided by a plurality of fingers 220′ proximate the end 214 opposite the flared end 216. Retention feature 120 is the same as discussed above for retention feature 20, and thus, no further description is needed. Outer surface 224 can be textured, and inner surface 228 can include a sanitizer 232, if desired, as discussed above. In contrast to the outer surface 124, the outer surface is not cylindrical, but rather, the outer surface 224 has a flat surface 38 extending from one of the ends 214 to the other of the ends 216, wherein the constant radius portion 234, relative to axis A, extends from one lengthwise extending side 40 of the flat surface 38 to an opposite lengthwise extending side 42 of the flat surface 38. The flat surface 38 facilitates marking the cable organizer 210, such as by writing on or applying a label to flat surface 38. Otherwise, cable organizer 210 is the same as cable organizer 110.

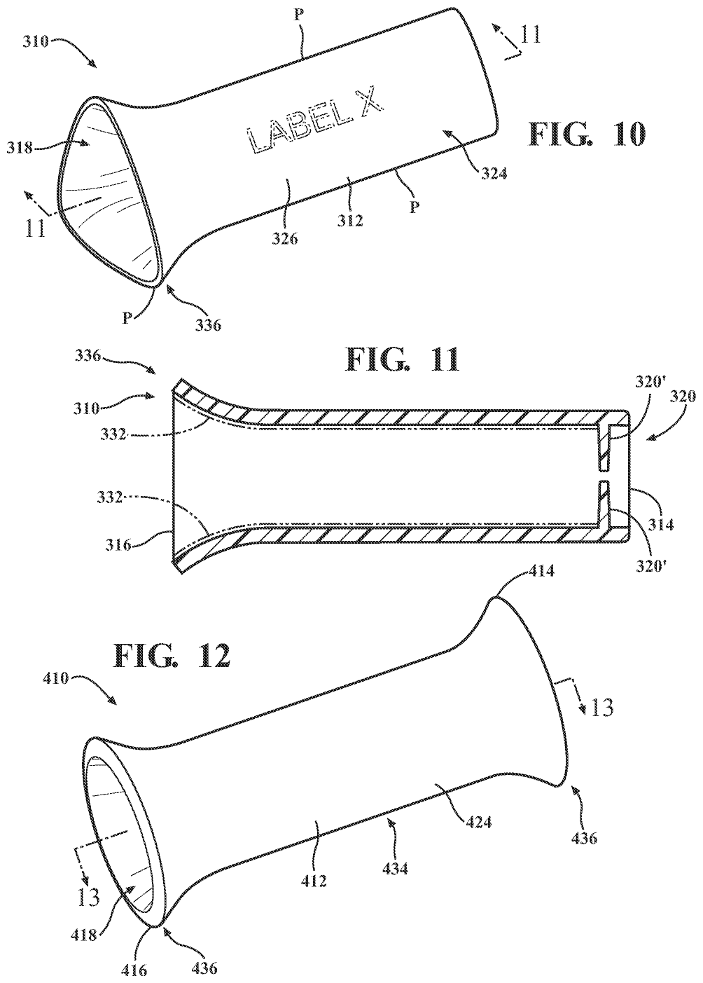

In FIGS. 10 and 11, a cable organizer 310 constructed in accordance with another aspect of the disclosure is shown, wherein the same reference numerals as used above, offset by a factor of 300, are used to identify like features.

Cable organizer 310 is similar to cable organizer 10, having an annular wall 312 extending along a longitudinal axis A (FIG. 11) between opposite ends 314, 316. The wall 312 has an outer surface 324 and an inner surface 328 that bounds a cavity 318, which extends from one end 314 to the opposite end 316. The outer surface 324 has a generally triangular outer surface 324, with convex surfaces 326 extending between three peaks P. A flared portion 336 extends from the ends 316A opposite retention feature 320. Retention feature 320 is provided by a plurality of fingers 320′ proximate the end 314 opposite end 316. Retention feature 320 is the same as discussed above for retention feature 20, and thus, no further description is needed. Outer surface 324 can be textured, and inner surface 328 can include a sanitizer 332, if desired, as discussed above. Otherwise, cable organizer 310 is the same as cable organizer 10.

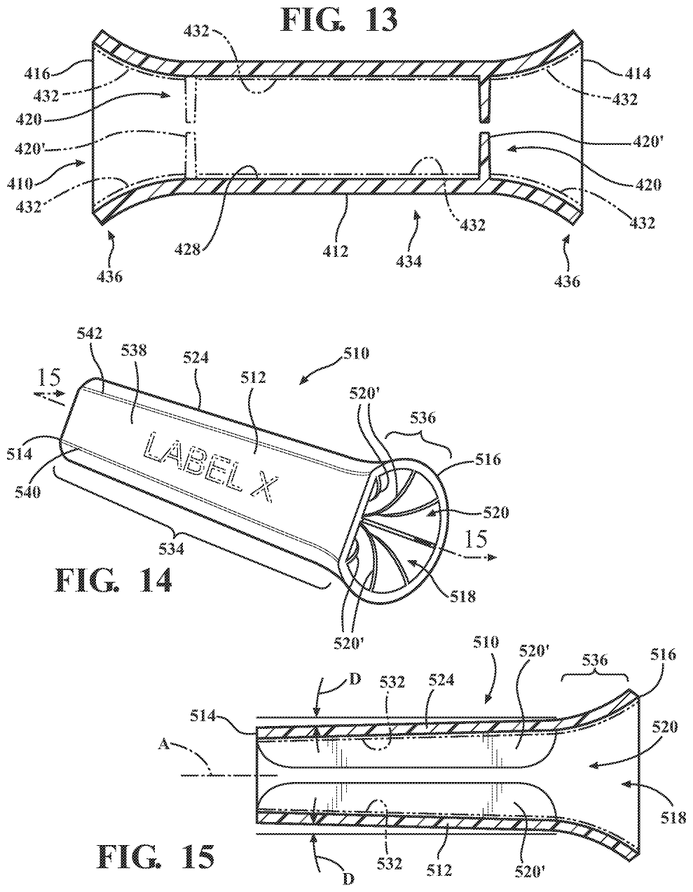

In FIGS. 12 and 13, a cable organizer 410 constructed in accordance with another aspect of the disclosure is shown, wherein the same reference numerals as used above, offset by a factor of 400, are used to identify like features.

Cable organizer 410 has an annular wall 412 extending along a longitudinal axis A (FIG. 13) between opposite ends 414, 416, as discussed above for wall 112. The wall 412 has an outer surface 424 and an inner surface 428 that bounds a cavity 418, which extends from one end 414 to the opposite end 416. The outer surface 424 has a constant radius portion 434 extending between the ends 414, 416. Flared portions 436, as discussed above for flared portion 136, extend from the ends 414, 416 to the constant radius portion 434. The constant radius portion 434 is cylindrical. A retention feature 420 is provided by a plurality of fingers 420′ proximate at least one end 414 (shown in solid) or each (420′ proximate flared end 416 shown in phantom) flared end 414, 416. Retention features 420 are the same as discussed above for retention feature 20, and thus, no further description is needed. Flared ends 436 facilitate gripping and moving cable organizer 410 in opposite directions along a cable bundle. Outer surface 424 can be textured, and inner surface 428 can include a sanitizer 432, if desired, as discussed above.

In FIGS. 14 and 15, a cable organizer 510 constructed in accordance with another aspect of the disclosure is shown, wherein the same reference numerals as used above, offset by a factor of 500, are used to identify like features.

Cable organizer 510 is similar to cable organizer 210, having an annular wall 512 extending along a longitudinal axis A (FIG. 15) between opposite ends 514, 516. The wall 512 has an outer surface 524 and an inner surface 528 that bounds a cavity 518, which extends from one end 514 to the opposite end 516. The outer surface 524 has a constant radius portion 534 extending from one of the ends 514 toward the other of the ends 516 and a flared portion 536 extending from the other of the ends 516 to the constant radius portion 534. A retention feature 520 is provided by a plurality of fingers 520′. Retention feature 520, rather than having coplanar fingers, is provided by fingers 520′ extending axially in generally parallel relation with the axis A between the opposite ends 514, 516. Outer surface 524 can be textured, and inner surface 528 can include a sanitizer 532, if desired, as discussed above. Outer surface 524 has a flat surface 538 extending from one of the ends 514 to the other of the ends 516, wherein the constant radius portion 534 extends from one lengthwise extending side 540 of the flat surface 538 to an opposite lengthwise extending side 542 of the flat surface 538. Otherwise, cable organizer 510 is the same as cable organizer 210.

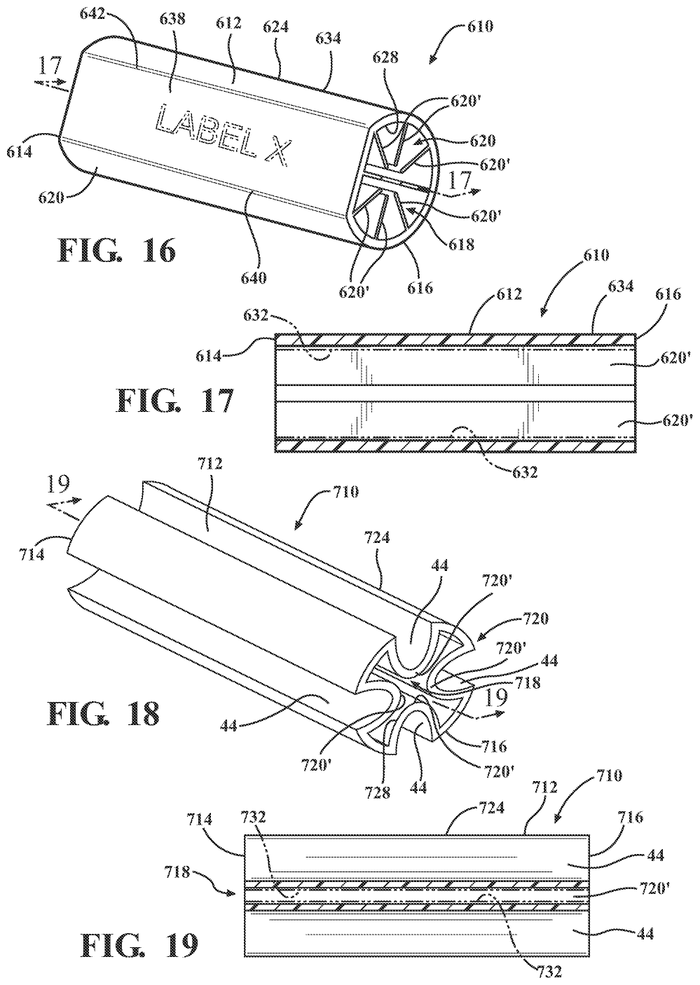

In FIGS. 16 and 17, a cable organizer 610 constructed in accordance with another aspect of the disclosure is shown, wherein the same reference numerals as used above, offset by a factor of 600, are used to identify like features.

Cable organizer 610 is similar to cable organizer 510, having an annular wall 612 extending along a longitudinal axis A (FIG. 17) between opposite ends 614, 616. The wall 612 has an outer surface 624 and an inner surface 628 that bounds a cavity 618, which extends from one end 614 to the opposite end 616. The outer surface 624 has a constant radius portion 634 extending from one of the ends 614 to the other of the ends 616. A retention feature 620 is provided by a plurality of fingers 620′. Retention feature 620 is provided by fingers 620′ extending axially in generally parallel relation with the axis A between the opposite ends 614, 616. Outer surface 624 can be textured, if desired, and inner surface 628 can include a sanitizer, as discussed above. Outer surface 624 has a flat surface 638 extending from one of the ends 614 to the other of the ends 616, wherein the constant radius portion 634 extends from one lengthwise extending side 640 of the flat surface 638 to an opposite lengthwise extending side 642 of the flat surface 638. The aforementioned features of cable organizer 610 have an axially extending symmetry that can be readily produced via an extrusion or pultrusion process, thereby being economical in manufacture. Otherwise, cable organizer 610 is the same as cable organizer 510.

In FIGS. 18 and 19, a cable organizer 710 constructed in accordance with another aspect of the disclosure is shown, wherein the same reference numerals as used above, offset by a factor of 700, are used to identify like features.

Cable organizer 710 has a circumferentially continuous, tubular wall 712 extending along a longitudinal axis A (FIG. 19) between opposite ends 714, 716, as discussed above for the previous embodiments. The wall 712 has an outer surface 724 and an inner surface 728 that bounds a cavity 718, which extends from one end 714 to the opposite end 716. The outer surface 724 has a plurality of constant radius portions 734 extending from one of the ends 714 to the other of the ends 716. The constant radius portions 734, relative to the axis A, are spaced circumferentially from one another by generally U-shaped troughs 44 that extend radially inwardly toward the axis A. A retention feature 720 is provided by a plurality of fingers 720′, wherein the fingers 720′ extend axially in generally parallel relation with the axis A between the opposite ends 714, 716. The plurality of fingers 720′ are formed via troughs 44, and thus, the fingers 720′ are generally U-shaped, as viewed looking along the axis A. The fingers 720′ are resiliently deflectable radially outwardly from the axis A upon disposing cable(s) through the cavity 718, whereupon the fingers 720′ engage an outer surface of the cables to maintain the cable organizer 710 in releasably fixed relation relative to the cables, regardless of the orientation of the cables, as is the case with each previous embodiment. Outer surface 724 can be textured, and inner surface 728 can include a sanitizer 732, if desired, as discussed above. The aforementioned features of cable organizer 710 have an axially extending symmetry that can be readily produced via an extrusion or pultrusion process, thereby being economical in manufacture.

In accordance with a further aspect of the disclosure, a method of organizing and/or identifying cables 11 of a medical apparatus 13 is provided. The method includes disposing at least one cable 11 through a cavity of a tubular wall 12, 112, 212, 312, 412 of a cable organizer 10, 110, 210, 310, 410 and bringing a retention feature 20, 120, 220, 320, 420 extending radially inwardly from the tubular wall toward a central axis A of the tubular wall into engagement with the at least one cable, wherein the retention feature deflects resiliently along the central axis A as the at least one cable 11 is being disposed through the cavity and inhibits relative movement between the at least one cable 11 and the cable organizer upon locating the cable organizer 10, 110, 210, 310, 410 along the at least one cable 11.

In accordance with a further aspect, the method can include, among other things discussed above, bringing a cleaning agent 30 disposed on an inner surface of the tubular wall 12, 112, 212, 312, 412 into engagement with the at least one cable 11 to sanitize the at least one cable 11 as the at least one cable 11 and the cable organizer 10, 110, 210, 310, 410 are being moved relative to one another.

This written description uses examples to disclose the embodiments, including the best mode, and also to enable those of ordinary skill in the art to make and use the invention. The patentable scope is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

Note that not all of the activities described above in the general description or the examples are required, that a portion of a specific activity may not be required, and that one or more further activities can be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

It can be advantageous to set forth definitions of certain words and phrases used throughout this patent document. The term “communicate,” as well as derivatives thereof, encompasses both direct and indirect communication. The terms “include” and “comprise,” as well as derivatives thereof, mean inclusion without limitation. The term “or” is inclusive, meaning and/or. The phrase “associated with,” as well as derivatives thereof, can mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items can be used, and only one item in the list can be needed. For example, “at least one of: A, B, and C” includes any of the following combinations: A, B, C, A and B, A and C, B and C, and A and B and C.

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended claims or claim elements unless the exact words “means for” or “step for” are explicitly used in the particular claim, followed by a participle phrase identifying a function.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. As used herein, the terms “substantial” and “substantially” means, when comparing various parts to one another, that the parts being compared are equal to or are so close enough in dimension that one skill in the art would consider the same. Substantial and substantially, as used herein, are not limited to a single dimension and specifically include a range of values for those parts being compared. The range of values, both above and below (e.g., “+/−” or greater/lesser or larger/smaller), includes a variance that one of skill in the art would know to be a reasonable tolerance for the parts mentioned.

Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or essential feature of any or all the claims.

After reading the specification, skilled artisans will appreciate that certain features which, for clarity, are described herein in the context of separate embodiments, can also be provided in combination in a single embodiment. Conversely, various features that are, for brevity, described in the context of a single embodiment, can also be provided separately or in any subcombination. Further, references to values stated in ranges include each and every possible value within that range.

What is claimed is:

1. A cable organizer for bundling and/or identifying one or more cables, comprising:
a wall having an outer surface and an inner surface bounding an inner cavity extending along an axis between opposite ends of said wall;
at least one retention feature extending radially inwardly from said inner surface toward said axis, said at least one retention feature including a plurality of fingers extending from said inner surface radially inwardly to free ends.

2. The cable organizer of claim 1, wherein each finger of said plurality of fingers is spaced from an adjacent one of said plurality of fingers by a gap.

3. The cable organizer of claim 1, wherein said plurality of fingers are coplanar.

4. The cable organizer of claim 3, wherein said plurality of fingers are resiliently flexible in opposite axial directions along said axis.

5. The cable organizer of claim 4, wherein said wall has a wall thickness and said plurality of fingers have a finger thickness, said finger thickness being less that said wall thickness.

6. The cable organizer of claim 1, wherein said outer surface has a generally triangular shape, as looking along said axis, defined by three wall sections, wherein said plurality of fingers includes three fingers, wherein a separate one of said three fingers extends radially inwardly from a separate one of said three separate wall sections.

7. The cable organizer of claim 6, wherein the outer surface each of said three wall sections is convex along a circumferential direction.

8. The cable organizer of claim 6, wherein the outer surface has an annular flared portion extending from one of said opposite ends toward the other of said opposite ends.

9. The cable organizer of claim 1, wherein the outer surface is tapered from one of said ends to the other of said ends, such that the outer maximum radial dimension of said one end is less than the outer maximum radial dimension of said other end, wherein the retention feature is proximate said one end.

10. The cable organizer of claim 1, wherein the at least one retention feature includes a pair of retention features with a separate one of said pair of retention features being proximate a separate one of said opposite ends.

11. The cable organizer of claim 1, further including a cleaning agent extending along at least a portion of said inner surface.

12. The cable organizer of claim 1, wherein said outer surface has a constant radius portion extending from one of said ends toward the other of said ends, and a flared portion extending from said other of said ends to the constant radius portion.

13. The cable organizer of claim 12, wherein said outer surface has a flat surface extending from said one of said ends to the other of said ends, said constant radius portion extending from one side of said flat surface to an opposite side of said flat surface.

14. The cable organizer of claim 1, wherein said plurality of fingers extend axially in generally parallel relation with said axis between said opposite ends.

15. The cable organizer of claim 14, wherein said plurality of fingers are generally U-shaped, as viewed looking along said axis, said plurality of fingers being resiliently deflectable radially outwardly from said axis.

16. A system for organizing and identifying cables extending from a source to a target, the system comprising:
a plurality of cable organizers, each cable organizer configured to slidably and releasably engage a subset of the cables simultaneously, such that each cable organizer is freely movable along a length of the subset of the cables; and
a cleaning agent on an inner surface of each cable organizer configured to sanitize the cables.

17. The system of claim 16, wherein at least some of the cable organizers differ from each other by at least one physical parameter.

18. The system of claim 17, wherein the physical parameter is at least one of color, shape, size or texture.

19. A method of organizing and/or identifying cables of a medical apparatus, comprising:
disposing at least one cable through a cavity of a tubular wall of a cable organizer; and
bringing a retention feature extending radially inwardly from the tubular wall toward a central axis of the tubular wall into engagement with the at least one cable, wherein the retention feature deflects resiliently along the axis as the at least one cable is being disposed through the cavity and inhibits relative movement between the at least one cable and the cable organizer upon locating the cable organizer along the at least one cable.

20. The method of claim 19, further including bringing a cleaning agent disposed on an inner surface of the tubular wall into engagement with the at least one cable to sanitize the at least one cable as the at least one cable and the cable organizer are being moved relative to one another.
